(19) United States
(12) Patent Application Publication
(10) Pub. No.: US 2014/0012144 A1
(43) Pub. Date: Jan. 9, 2014

(54) PERFUSION DETECTION SYSTEM

(71) Applicant: William E. CRONE, FALL CITY, WA (US)

(72) Inventor: William E. CRONE, FALL CITY, WA (US)

(21) Appl. No.: 13/937,507

(22) Filed: Jul. 9, 2013

Related U.S. Application Data
(60) Provisional application No. 61/733,871, filed on Dec. 5, 2012.
(60) Provisional application No. 61/733,865, filed on Dec. 5, 2012.
(60) Provisional application No. 61/669,474, filed on Jul. 9, 2012.

(51) Int. Cl. A61B 5/02; A61B 7/00; A61B 5/00; A61B 5/024; A61B 5/11; A61B 5/046

(52) U.S. Cl. CPC A61B 5/02028; A61B 5/11; A61B 5/046; A61B 5/746; A61B 5/02416; A61B 7/00
USPC 600/483; 600/481; 600/508

(57) ABSTRACT

According to some embodiments, a system for detecting a perfusion index of a cardiac pulse includes a first sensor that senses a first physiological or environmental parameter of a human patient core, a second sensor that senses a second physiological or environmental parameter of the human patient core, a processor that, responsive to the first and second sensed parameters, determines a perfusion index ranging from 0 to 10 that reflects inadequate, marginal, or adequate blood perfusion to the core of the human patient torso, and an indicator that provides a discernible indication of the perfusion index. A method of detecting as perfusion index of a cardiac pulse responsive to sensing first and second physiological or environmental parameters of a human patient core is also disclosed.

### CLAIM OF PRIORITY

The present application claims the benefit of U.S. Provisional Patent Applications Ser. No. 61/669,474, filed Jul. 9 2012; Ser. No. 61/733,865, filed Dec. 5, 2012; and Ser. No. 61/733,871, filed Dec. 5, 2012 all of which applications are incorporated herein by reference in their entireties.

### BACKGROUND

The ability to determine the existence of sufficient oxygenated blood to the head and neck to keep a patient from suffering permanent cognitive damage is of key importance in multiple situations. EMS (Emergency Medical services) professionals and primary care physicians have long sought for a device that can quickly assess the existence of a perfusion pulse to assist them in determining the proper immediate action necessary for the preservation of cognitive neurological function.

In addition the ability to have physiological feedback while performing emergency CPR is also of key importance to insure the highest probability of the patient being neurologically intact after various types of cardiopulmonary events.

As an example, the ability to determine that a human subject is in PEA (Pulseless Electrical Activity) is key as the method of treatment may be different than if the patient was determined to be in Ventricular Fibrillation.

The number of people worldwide that are impacted by sudden cardiac death (SCD) ranges from the hundreds of thousands per year to numbers that exceed 1 million depending on the literature sited. Embodiments described herein and associated methodology to achieve the above performance is aimed at assisting the lay person, the EMS professional and the entire medical community in determining the immediate and long term assurance of adequate perfusion.

Reliable perfusion detection, the ability to determine that there is a “pulse” is difficult and has been attempted by numerous colleagues. The problem relates to determining in the presence of motion, various pharmocologics, and external environmental contributors, when the patient has an adequate pulse to keep them alive without cognitive damage until medical professionals arrive. Embodiments described herein address these and other issues by providing a solution through use of physiological parameters and additional body worn sensors.

### SUMMARY

Embodiments use multiple physiological and non physiological sensors to determine the presence of a pulse. A Photoplethsmography waveform describes the time related absorption of light associated with Oxy and DeOxy hemoglobin. Analysis of the photoplethsmography contour may be evaluated to assist in the indirect assessment of stroke volume and cardiac output. In addition, usage of a three axis accelerometer/force sensor, acoustics, as for example, particular heart sounds associated with the closure of the aortic valve S2, and the ECG (electrocardiogram) may be utilized to determine an adequate perfusion pulse. The parameter that is presented is called the ‘perfusion index’. The perfusion index is a numeric value that relates the degree of perfusion to various organs in the body. The higher the number the higher the perfusion to the vital organs in the body. The number is relative and values range from 0 to 10. The level deemed ‘adequate’ to prevent neurological damage to the patient is determined by a preset value determined by numerous clinical trials on various patients with various life threatening conditions.

Usage of a single physiological parameter such as photoplethsmography is prone to motion sensitivity, application pressure, and sensor location.

Embodiments described herein utilize multiple physiological parameters to determine the existence of a perfusion pulse using the core homeostasis of the human. No peripheral locations are utilized. While peripheral locations could be utilized they are more prone to errors associated with peripheral vascular disease, shock, and other conditions where the body has decided to shut down the peripheral system in order to maintain the core. Multiple parameters are utilized to reduce the typical confounding variables associated with accurate detection of a pulse, such as motion, pressure, acceleration/force, and location.

To determine that a perfusion pulse exists to the brain it is necessary to determine that there is sufficient stroke volume to move oxygenated blood from the left ventricle of the heart muscle to the head within a specified period of time. To do this a trigger may start a time clock to determine how may milliseconds after a compression (Heart systole or external chest compression)(CPR-CardioPulmonary Resuscitation) the signal arrives at a location in the core of the human subject. To do this the trigger to start the time can be the QRS complex of the ECG (electrocardiogram) if it is present, or it could be a signal from an accelerometer/force sensor (1 2, or 3 axis) which indicates the force of the heart when contracting (systolic period), or it could be a signal associated with external chest compression or heart sounds associated with the aortic valve closing (S2 sound). The evaluation of the photoplethsmography contour itself may be utilized to assist in the determination of the cardiac output/stroke volume. This is important as during manual heart compression (CPR) and in hemodynamically compromised patients the contour of the photoplethsmography waveform will be quite different than in an alert patient. Prior to evaluating the contour of the waveform the waveform is preferably normalized. The accelerometer/force sensor can be located in a multiple of places on the human torso but are typically placed on the sternum during CPR, on the head, or in a wearable band placed below the nipples on the chest. If the ECG is not present, then the accelerometer/force sensor and/or heart sounds can be utilized to determine when the heart aortic valve opens to eject blood from the left ventricle. The time taken from the time the aortic valve opens to the time the photoplethsmography pulse arrives at a predefined location on the core of the human subject can be utilized. The pulse is defined as an increased volume of blood associated with the stroke volume of the patient's heart. In cases where heart sounds are not detectable and the ECG is not present, the accelerometer force sensor that detects motion associated with CPR can be used as the timing trigger.

The calculation of the timing between the accelerometer/force sensor signal and/or the ECG signal and the arrival of the photoplethsmography signal, may be modified using contour analysis of the photoplethsmography normalized waveform.

The pre-ejection time, the time period between the QRS complex and the time the blood is ejected through the aorta can be measured in those cases where heart sounds are available, and then placed into a regression formula along with the time from the detected QRS complex to the arrival of the normalized contour modified amplitude photoplethsmography detection point at a prescribed location on the human torso. The detection point of the photoplethsmography signal may be adjusted as a function of the contour of the photoplethsmography waveform and its first derivative. The resultant output of the regression formula is correlated to the valid range of time periods and a decision is made as to whether the timing is consistent with adequate perfusion. A “perfusion index” is calculated and if the time period is beyond, longer than the lower time limit, the preset value the system activates an alarm one of the key benefits of this perfusion system is the use of at least two physiological parameters to determine an adequate perfusion index and time frequency relationship between them. While this perfusion detection system has the highest specificity and sensitivity when used with the ECG, an Accelerometer/force sensor, Photoplethsmography contour, and acoustic sensors, determination of the perfusion index can be achieved with as little as two physiological sensors, albeit with lower sensitivity and specificity that if all sensors were available.

Hence, according to some embodiments, a system for detecting a perfusion index of a cardiac pulse comprises a first sensor that senses a first physiological or environmental parameter of a human patient core, a second sensor that senses a second physiological or environmental parameter of the human patient core, a processor that, responsive to the first and second sensed parameters, determines a perfusion index ranging from 0 to 10 that reflects inadequate, marginal, or adequate blood perfusion to the core of the human patient torso, and an indicator that provides a discernible indication Of the perfusion index.

The first and second sensors may include at least one of an accelerometer/force sensor, photoplethsmography sensor, an ECG sensor, one or more leads, a one to three axis accelerometer/force sensor, and an S1/S2 heart sound sensor. The system may further include a cardiac arrest detector. The system may still further include a detector that detects the existence of PEA (Pulseless electrical activity).

The processor may be programmable to determine the presence of atrial fibrillation for those patients who have low EF (Ejection Fraction).

According to other embodiments, a method of detecting a perfusion index of a cardiac pulse comprises sensing a first physiological or environmental parameter of a human patient core, sensing a second physiological or environmental parameter of the human patient core, determining, responsive to the first and second sensed parameters, a perfusion index ranging from 0 to 10 that reflects inadequate, marginal, or adequate blood perfusion to the core of the human patient torso, and providing a discernible indication of the perfusion index.

### BRIEF DESCRIPTION DRAWINGS

FIG. 1 is a graph showing an idealized response of an accelerometer/force sensor or force sensor to the recoil associated with the forces of the heart.

FIG. 2 is a graph showing an idealized ECG waveform showing the QRS complex used to for a trigger in the timing described in this application.

FIG. 3 is a graph showing idealized heart sounds heard through a stethoscope, microphone, or other acoustic devices. The S1 S2 sounds are associated with various valves closing during the cardiac cycle, the ‘lub dub’ as it is often referred to literature.

FIG. 4 is a graph of an inverted photoplethsmography signal taken at one of many isosbestic wavelengths. This waveform would look approximately the same if taken at one of the many IR wavelengths often used when determining SpO2.

FIG. 5 is a block diagram of a system for practicing embodiments of the invention.

FIG. 6 is a flow chart describing the steps which may be taken by the system of FIG. 5 to determine if a person has adequate perfusion to prevent neurological damage to the brain and other organs in the body.

### DETAILED DESCRIPTION

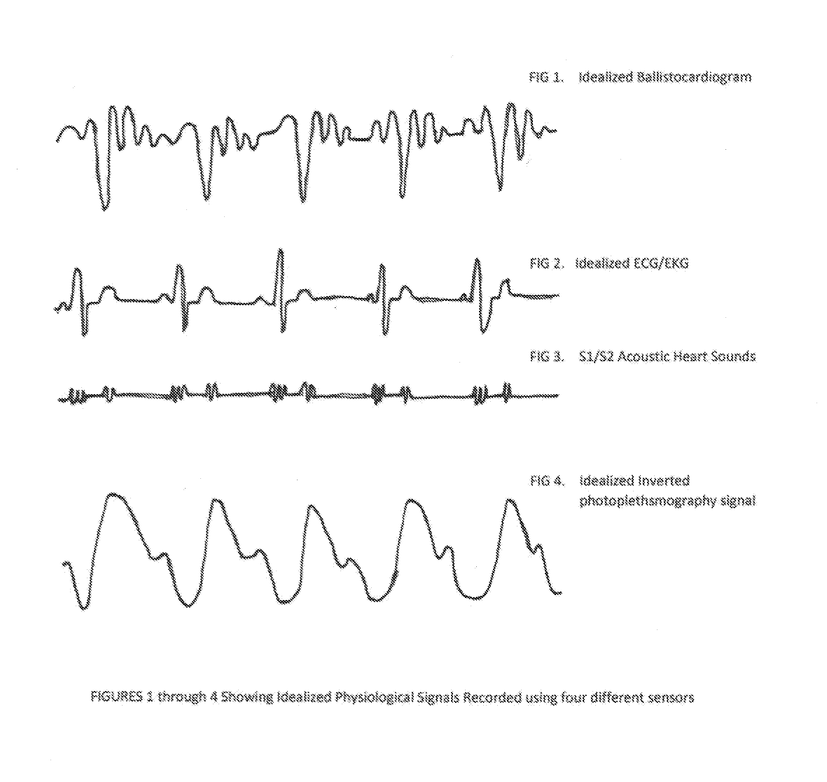

FIGS. 1-4 are a time combined set of waveforms showing the various sensors and associated waveforms that may he utilized in practicing the invention in its various embodiments to advantage. FIG. 1 is an idealized response of an accelerometer/force sensor or force sensor to the recoil associated with the forces of the heart. FIG. 2 is an idealized ECG waveform showing the QRS complex used to form a trigger in the timing described herein. FIG. 3 are idealized heart sounds heard through a stethoscope, microphone, or other acoustic devices. The S1 S2 sounds are associated with various valves closing during the cardiac cycle, the ‘lub dub’ as it is often referred to literature. FIG. 4 Shows an inverted photoplethsmography signal taken at one of many isosbestic wavelengths. This waveform would look approximately the same if taken an one of the many IR wavelengths often used When determining SpO2.

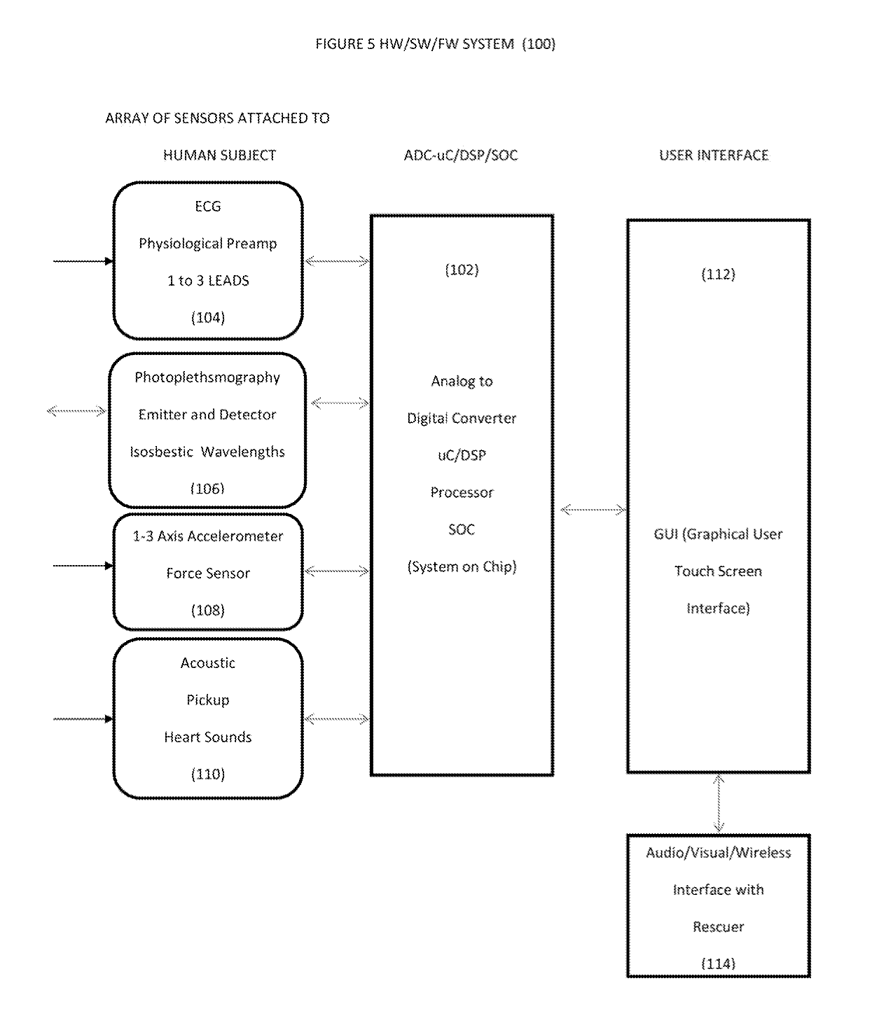

Referring now to FIG. 5, it is a block diagram of a system according to embodiments of the invention. The system 100 generally includes a processor or microcontroller (uC) 102 and various peripheral circuits or units to generate data or display data and/or notifications to an operator. The uC 102 is arranged to operate according to operating instructions stored in memory. The operating instructions permit the uC to perform analog to digital conversion, processing of data to determine the various parameters disclosed herein, and to function as a wireless transceiver.

The various circuits or units include a physiological preamp with 1 to 3 leads 104, a photoplethsmography emitter and detector 106, a 1-3 axis accelerometer and force sensor 108, and an acoustic sensor 110 to pickup heart sounds. The circuits and units further include a graphic user touch screen interface 112 and an audio/visual wireless interface. As will be seen, programming the uC in various ways permit the system to function as a perfusion detector, a tool to assist the operator in performing CPR, an atrial fibrillation detector, a detector for pulseless electrical activity (PEA), a detector for low ejection fraction, and to determine Asystole. Further, the uC 102 may be programmed with parameter ranges to enable various required comparisons to determine if various parameters are within certain ranges. Other functions of the uC and of the system 100 will become apparent herein after.

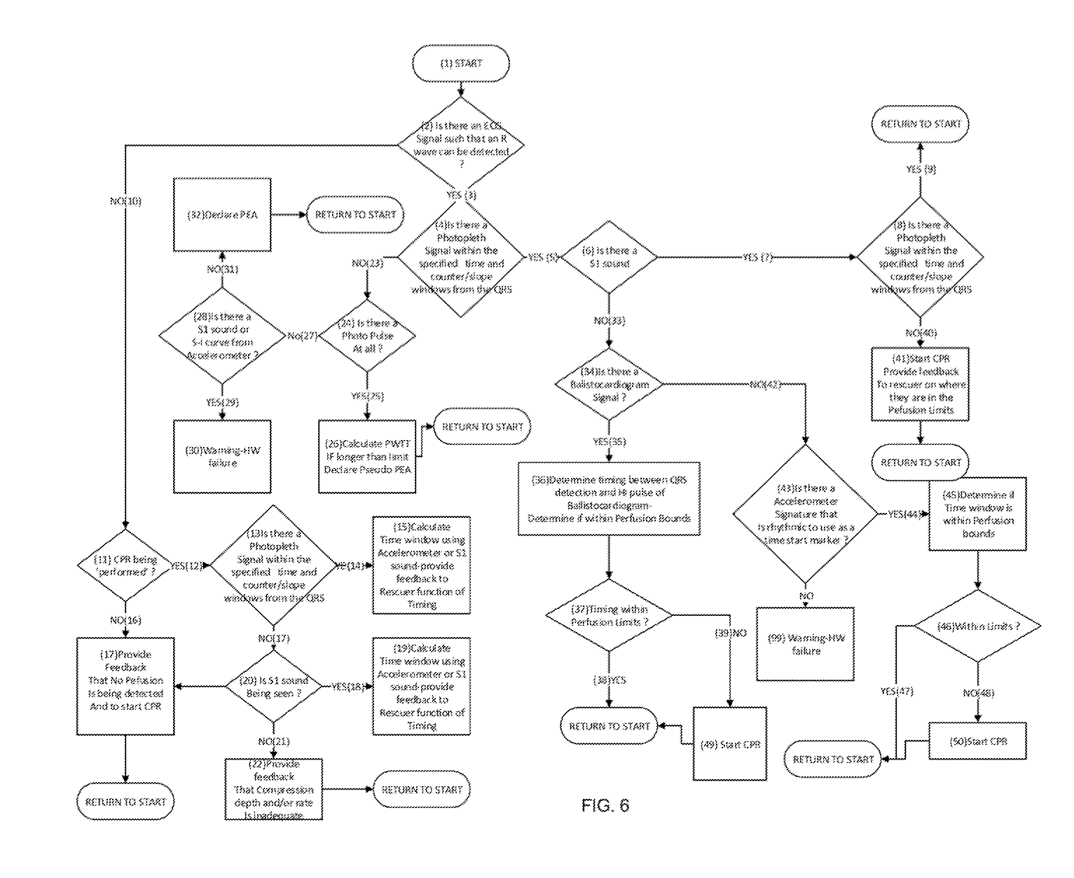

Referring now to the flow chart of FIG. 6, upon Power On, the uC of the system performs some rapid self tests and then the process proceeds down the flow chart.

The first, determination made in decision block 2 is if there an ECG Signal such that an R wave can be detected. This processing is done by sampling the surface ECG at one or more locations on the core of the human subject. This location could be the forehead, the chest, or other locations such as the common ‘limb leads’ used as in the case of a standard single, 3, 5, or 12 lead ECG.

The ECG is sampled with sufficient bandwidth to allow for the detection of the QRS of the ECG waveform even in the presence of an internal or external pacemaker. The method used to detect the QRS may consist in part of digital filtering of the ECG to reduce the signal levels of the P wave, T wave, triboelectric interference, motion, and various other external confounding variables. After analog filtering and digitization of the ECG waveform, digital adaptive filters along with wavelet transformations are used to determine the QRS location in the presence of the remaining confounders. Environmental noise, motion, including CPR, and RFI (Radio Frequency Interference)/EMI (Electromagnetic Interference) are some typical examples.

If the decision in decision block 2 is NO (10) then the process proceeds to decision block 11 where it is determined if CPR is being performed. By examining the output of the three axis accelerometer/force sensor the rhythmic pattern of CPR can be detected. The method used for this determination may utilize Wavelet transformations and Gabor Spectrograms to extract the time/frequency signature of CPR in the presence of head and torso movement associated CPR.

If it is determined that no CPR is detected, then, in accordance with activity block 17, feedback is provided to the rescuer through the decision matrix of the uC(microcontroller) that no CPR nor perfusion has been detected and to start CPR. The process the returns.

If CPR was detected in decision block 11, then the system, in accordance with decision block 13 evaluates the presence of a photoplethsmography signal and determines if the change in volume measured by the photolethsmography system is consistent in time with the QRS complex to represent sufficient perfusion to sustain the patient. If there is a photoplethsmography signal available, then the process proceeds to activity block 15 where the timing between the CPR compression and the arrival of the increased blood volume at the photoplethsmography site is performed. In conjunction with the accelerometer/force sensor detection of the Chest compression, the detection of the S1/S2 heart sounds could be utilized in conjunction with or in lieu of the accelerometer/force sensor signature. A photoplethsmography signal at various wavelengths can be used but in this system choose to use isosbestic wavelengths to remove any confounding variables associated with the level of oxy/deoxy hemoglobin levels associated with the patient's blood. By examining the photoplethsmography signal and its contour and looking for the reduction in signal level associated with the largest volume of blood that passes the isosbestic light source and corresponding optical receiver array, a determination can be made of the time taken to move the large volume of blood from the Left ventricle to the position of the sensor, the change in volume associated with Left Ventricular contraction. After securing this time value a decision is made as to whether the arrival of the large volume of blood is within a preset value range. This information is then used to provide feedback to the rescuer as to the adequacy of their compressions. If the time period calculated is long the rescuer is encouraged to push harder and faster. The determination of whether the depth is inadequate and/or the compression rate is too slow is determined by calculating the compression rate using the 1-3 axis accelerometer. If the time period is short then the feedback that is provided is that the CPR is being performed well.

If the Photoplethsmography signal, the increased blood volume time arrival at the photoplethsmography site is too long, then the decision of decision block 13 is NO and the existence of an S1/S2 sound is evaluated. This is performed in decision block 20 where an S1/S2 sound is determined to exist. Then the required time interval between the S1/S2 sound and the arrival of the increased blood volume is calculated in activity block 19. If no S1/S2 Sound is found in decision block 20, then, in activity block 22 feedback is provided to the rescuer that no adequate perfusion is being found and that CPR needs to be initiated or compression depth/rate needs to increase. If the time window calculated using the S1/S2 sound and the arrival of the increased blood volume passing through the optical sensors are within the specified range, then the decision in decision block 20 is YES (18) and the corresponding perfusion index is calculated and fed back to the rescuer in activity block 19. The perfusion index in this decision sequence is determined using the detection of a CPR Compression and the time to detection of the increase in blood volume at the optical sensor array position.

If in decision block 22 a QRS complex is detected, then the process proceeds to decision block 4 to determine if a photoplethsmography signal representing increased blood volume within a specified time window from the QRS complex of the ECG is present. The detection of increased blood volume may be detected by the decrease in optical light, detected at the optical receiver array due to the increase in absorption of the specific wavelength of light associated with blood. The derivative of the optical waveform is performed to include in the decision process the contour of the photoplethsmography waveform, If the decision of decision block 4 is YES, then the existence of S1/S2 sounds representing closure of the atrial ventricular valves and the aortic valve are evaluated in decision block 6, if the answer to this decision in decision block 6 is YES, then, the time sequence from the QRS complex and the S1/S2 sounds and from the S1/S2 sounds to the Photoplethsmography signal is evaluated in decision block 8 and the corresponding perfusion index is calculated if the time between the S1/S2 sounds or the QRS complex is outside of bounds (a NO answer in decision block 8 then the rescuer is told to start CPR in activity block 41. If the perfusion calculation shows that the perfusion index is within specified range the decision in decision block 8 is YES, the rescuer is told that the patient has a viable perfusion index and provided with a numeric value for the displayed number. The process then returns.

If the decision in decision block 6 is that there is no S1/S2 sound, the existence of a Ballistocardiogram is determined in decision block 34. Here, the HI curve and/or the IJK curve can be determined to exist or not exist and can potentially be used to determine the perfusion index. The method for determining the HI curve of the Ballistocardiogram may use various descriptors including template matching of the HI, IJK curves, their derivatives, force sensor: the force of the contraction of the Left Ventricular ballistocardiogram, wavelet transforms and the Gabor spectrogram. If the Ballistocardiogram exists, in particular the HI and or IJK curves, then the process proceeds to activity block 36 where the perfusion index can be calculated by looking at the time period between the HI, IJK curves of the Ballistocardiogram and the arrival of the increased blood volume at the photoplethsmography optical receiver site. The process then proceeds to decision block 37 to determine if the time period is within the required time window to represent a viable perfusion index. If it is, then the rescuer is informed of the perfusion index value (38, and the process returns. If the time period is NOT within the required time window, the rescuer is told to start or enhance/start CPR and the process returns.

If in decision block 34 no balistocardiogram signal is found, then, in decision block 43 it is determined if there is a three axis accelerometer/force sensor signal and is analyzed for a signature that is rhythmic in nature to determine if it can be utilized as a time marker. Again utilization of the signature of rhythmic CPR may be utilized along with Wavelet transforms and the Gabor spectrogram to capture the HI, IJK curves or the Ballistocardiogram as compared to other confounding variables.

Please note that during CPR the nature of the HI, IJK curves will change. The HI, IJK curves will not have the same morphology during manual CPR than during regular NSR (Normal Sinus Rhythm). The coefficiants of the formula will account for the morphological differences.

If the answer in decision block 43 is YES, the perfusion index is analyzed in activity block 45 and then a determination is made in decision block 46 if the analysis shows the perfusion index to be within bounds to represent a perfusion pulse. If the calculation shows that the perfusion index is within bounds, then the rescuer is informed and the process returns. If the result is not within bounds, the rescuer is prompted to start CPR in activity block 50.

It should be noted that at decision block 43, where an accelerometer/force sensor signal that is rhythmic in nature is not found while a valid photoplethsmography signal is available, an indication of a hardware failure is so noted in activity block 99.

Returning to decision block 4, if in decision block 4 it is determined that there is no photoplethsmography signal that represents the required increase in blood volume within the specified window after ventricular systole a couple of unique additional decisions are made. A check is made to determine if the photoplethsmography signal is ‘flat’ (asytole) given no accelerometer/force sensor signals, if the answer to this question “Is there a photoplethsmography signal” in decision block 24 is NO, then a final check to see if there is an S2 heart sound is checked in decision block 28. If not, then PEA is declared to the rescuer in activity block 32. If an S2 sound is found, then a HW failure is flagged and the rescuer alerted ion activity block 30. The processor will self check on a period nature to see if the fault has been cleared.

If in decision block 24 it is decided that there is a photoplethsmography signal but one that fails the criteria defined in decision block 4, then the calculation of a perfusion index is initiated in activity block 26 with the full understanding that the value is out of range. This condition may represent Pseudo PEA and the rescuer is so informed. The process then returns.

The following are exemplary technical details of how various aspects of this invention achieve the final goal of determination of the perfusion index.

The ECG waveform is filtered, and the detection of the QRS complex with minimum group delay is performed. Evaluation of the QRS complex and utilizing the QRS complex as a timing trigger is done in a repeatable manner associated with the detection of the QRS complex for use as a time trigger point.

Photoplethsmography is utilized to determine the contour and arrival of the systolic blood volume as a result of the Left Ventricle contraction and ejection of the stroke volume of blood on a beat by beat basis. The oxy and deoxy hemoglobin in the blood absorb light in the visual and infrared regions 300 nm to 2500 nm wavelengths and beyond. Specified wavelengths are selected that are close to the iosobestic wavelengths, 569, 805 to mention a few, but any wavelength can be used where blood hemoglobin absorbs light. One must be careful in selecting the desired wavelength due to environmental contamination. Water absorbs light above around 1100 nm and beyond so care must be utilized in selection of wavelengths above the Si cutoff of 900 nm. Water wavelengths have been utilized to assist in removing motion artifact from the receive signal in the past.

The contour of the photoplethsmography signal is examined by taking the derivative of the waveform and comparing the resultant contour to that of an alert perfusion contour. A least squares comparison is done and the result of this comparison results in a specific value(s) in the polynomial regression formula to be made.

The accelerometer/force sensor signals can from three locations: X, Y, Z. The position of the patient is determined along with any rhythmic pattern of the accelerometer/force sensor consistent with desired patient status of supine when being examined. In addition to any ‘rhythmic pattern’ various components of a Ballistocardiogram are investigated, in particular the HI curve and the IJK curves. These curves are analyzed as are their first and second derivatives. This information assists in determining the coefficients for the regression formula which has as its independent variable time and the y variable as the ‘perfusion index’, a value from 0 to 10 on the x axis.

The Ballistocardiogram is first normalized and then the HI, IJK curves, including first and second derivatives, and there corresponding contours are evaluated and compared to known contours associated with normal ventricular contractions and those associated with CPR and cardiovascular disease, and timing relative to the QRS complex if it is available. The results of this analysis determines coefficients in the polynomial used to determine the Perfusion index as described above.

The heart sounds, in particular S1,S2 are evaluated to see if the stroke volume and the nature of the closing of the atrial-ventricular valves and the aortic valve are consistent with sufficient perfusion to cause the aortic valve to close within a preset time window. The result of this analysis again modifies the coefficients in the perfusion detection polynomial.

All of the above physiological/environmental sensors may be utilized to adjust the perfusion index polynomial to insure that the various parameters are weighted correctly when applying the ‘perfusion index’ value.

Upon power ON the SOC uC does a series of self checks including sequencing available sensors for use in the Polynomial that determines the Perfusion index output. The device preferably indicates to the rescuer to ‘stand clear’ and ‘not touch the patient’.

After a 3 second waiting period for the accelerometer/force sensor signal to go to ‘zero’, if the motion signal ceases, the uC/DSP looks at the ECG signal and determines if an R wave is present that meets the frequency and temporal characteristics of a normally conducted R wave. If the motion signal does not cease, the processor uses a more highly filtered signal along with Wavelet Gabor spectrographs to separate the desired accelerometer/force sensor signal from that of motion. Usage of Wavelet transformations and Gabor spectrographs are also used to separate the motion artifact from the desired QRS complex.

If a photoplethsmography signal exists the algorithm determines the time between the approximate QRS detection point and a specific trigger point of the photo-plethsomography signal. This detection point is Adjustable by the health care provider at the time of manufacture used on this time window and if the Ballistocardiogram detection system indicates their Ballistocardiogram exists, the HI and IJK amplitudes/contours and their first/second derivative must meet a specified amplitude and contour set of criteria. Again the results of the amplitude and contour of the accelerometer/force sensor signals determines the coefficients that are utilized in the polynomial used to determine the ‘perfusion index’ as described above.

Heart sounds are utilized, if available to determine the functioning of the atrial-ventricular valves and the aortic valve. The morphology of the S1/S2 heart sounds are examined to insure, adequate perfusion. Here again the S1/S2 heart sounds are analyzed in the time frequency domain and the result of this analysis are the values chosen for the coefficients in the perfusion index.

If heart sounds are not heard and if no Ballistocardiogram/accelerometer/force sensor signals are present the compute engine can still make a decision about the presence of a perfusion pulse if the time window between the detected QRS and the arrival of the corresponding photopleth signal is within a specified time window and morphology/contour indicative of a viable perfusion interval and indirect assessment of stroke volume exist.

If an ECG is present but no photoplethsmography signal is available the compute engine looks for a Ballistocardiogram signature to exist or a rhythmic compression signature. If either of these signals are present during a specified time window from the detected QRS, then a decision can also be made about the presence of a perfusion pulse. Usage of Wavelets and the Gabor spectrograph are used in conjunction with template matching associated with the signature of the accelerometer/force sensor to determine if a rhythmic compression is occurring.

If no QRS is detected the compute engine listens for S1/S2 hearts sound and then examines the presence of a photoplethsmography signal within a specified time of the S1/S2 heart sounds. If no photopleth signal exists as well as no detected QRS or Ballistocardiogram HI/IJK signatures or rhythmic accelerometer/force sensor signal consistent with chest compressions, the device determines that there is no viable perfusion and provides necessary feedback to the rescuer.

If there is an accelerometer/force sensor signal that meets specified amplitude and morphology contours and that are repeatable within a 60-150 compression per minute rate the device assumes that CPR is being given and that in the absence of a QRS that the accelerometer/force sensor signal can be utilized along with the ballistocardiogram, S1/S2 heart sounds, and photoplethsmography signal to determine the success of the CPR compressions to form a photoplethsmography signal with sufficient timing between the accelerometer/force sensor signal and the photoplethsmography signal, the S1/S2 sounds and the Ballistocardiogram signature. A decision may be made about perfusion viability without the presence of the Ballistocardiogram or the S1/S2 sound but with these additional physiological inputs the accuracy of the system can be enhanced.

The S1/S2 and presence of the HI, IJK curves are utilized together to enhance the accuracy of the polynomial in selecting the best coefficients of the polynomial used to calculate the perfusion index.

The system can determine with a subset of the total number of physiological and environmental parameters available as described above whether a perfusion pulse exists in the case of PEA (Pulseless electrical Activity), CPR, VT/VF and Asystole.

PEA is determined by looking at the ECG, photoplethsmography signal, the Ballistocardiogram, the accelerometer/torte sensor, and the S1/S2 sounds. The ballisocardiogram and the S1/S2 sounds are not needed for a determination of PEA. These parameters enhance the decision sensitivity and specificity by being present as well. PEA is determined by the presence of a ECG waveform in the absence of or in the presence of low perfusion.

Asystole is determined by looking for the absence of a detected QRS, the absence of a Ballistocardiogram, an S1/S2 sounds, and the absence of a photoplethsmography signature in the presence of a QRS complex.

VT/VF is determined by looking at the QRS width, its morphology, its rate, its rate variability, and the presence of a Ballistocardiogram, an S1/S2 sound, and the accelerometer/force sensor signature. Various templates of VT/VF along with Wavelet transforms and Gabor Spectrogram's are used to enhance the sensitivity and specificity of the VT/VF detection.

The sensitivity and specificity of the detection of VT/VF is enhanced by the presence of the Ballistocardiogram and the S1/S2 sounds but these parameters are not required to make this decision.

CPR Quality, the ability to have a perfusion pulse, is determined as a minimum by the accelerometer/force sensor signature associated with Sternum compression and the time relationship of a photoplethsmography signal and the morphology/contour of each signal and their derivatives. During CPR the motion of the chest is significant so the preprocessing of the S1/S2 heart sounds and the Ballistocardiogram can be utilized only in the condition that the signal quality meets specified signal to artifact criteria.

The ‘sensors’ may consist of one or more locations of ECG electrodes, a 1-3 axis accelerometer/force sensor, respiratory detection in the form of impedance pneumography or other means(photoplethsmography/ECG/strain gage), photodiode emitters and corresponding photodetectors for detection of the photoplethsmography signal in one or more locations, and finally acoustic pickup devices for the detection of heart sounds, in one or more locations.

The ECG is digitized and the QRS is detected using a software algorithm. The detection point is usually near the J point of the ECG waveform.

The 1 to 3 axle accelerometer/force sensor signals are digitized and have a useful bandwidth of 0.1 to 5 hertz.

The photoemitters are pulsed at a high frequency and the photodiode receivers signals are then demodulated. The extracted photoplethsmography waveform associated with changes in signal attenuation associated with the cardiac cycle, is then digitized. Prior to digitization the DC and AC components of the photoplethsmography signal are separated and gained differently prior to digitization. (AC component has a higher gain than the DC component). The component of the waveform is inverted and then its contour analyzed and through use of Wavelet transforms and the gabor spectrogram the proper timing detection point can be developed.

The acoustic pickup is preprocessed by band-limiting the pickup frequency range, amplifying the signal and then digitizing the resultant signal. The contour and the output of a Wavelet transform and Gabor spectrogram are used to develop a proper select point of the S1/S2 sounds for determining the timing to be used in the polynomial for the perfusion detection algorithm. The timing between the acoustic pickup and the photoplethsmography waveform is critical for accurate assessment of the perfusion index.

As may be seen from the foregoing, embodiments described and shown herein provide a system and method capable of detecting a perfusion index of a cardiac pulse using two or more physiological and environmental signals received from the human core torso. The perfusion index is a numeric value ranging from 0 to 10 that reflects inadequate, marginal, or adequate blood perfusion to the core of the human torso.

Also described and shown is a system and method capable of detecting the perfusion of a cardiac pulse during cardiac arrest using two or more physiological and environmental signals received from the human torso and a system and method capable of detecting the existence of PEA (Pulseless electrical activity).

The system may utilize one or more of the following parameters to determine the presence of a perfusion index; an accelerometer/force sensor, photoplethsmography sensors, the ECG, one or more leads, a one to three axis accelerometer/force sensor, and S1/S2 sounds of the heart.

Also shown and described is a system and method with the ability to be programmed to determine the presence of AF (atrial fibrillation) for those patients who have low EF (Ejection Fraction).

The system also has the ability to determine Asystole.

Also, embodiments of the method and system may determine the proper polynomial coefficients for an equation that describes the perfusion index from a value range of 0 to 10 where the independent variable is time and the dependent variable is the perfusion index. The coefficients are dynamic and a function of the ECG, photoplethsmography wavform, the accelerometer/force sensor waveforms, and the acoustic S1/S2 when available.

While particular embodiments of the present invention have been shown and described, modifications may be made, and it is therefore, intended in the appended claims to cover all such changes and modifications which fall within the true spirit, and scope of the invention as defined by those claims.

What is claimed is:

1. A system for detecting a perfusion index of a cardiac Pulse Comprising:
a first sensor that senses a first physiological or environmental parameter of a human patient core
a second sensor that senses a second physiological or environmental parameter of the human patient core;
a processor that, responsive to the first and second sensed parameters, determines a perfusion index ranging from 0 to 10 that reflects inadequate, marginal, or adequate blood perfusion to the core of the human patient torso; and
an indicator that provides a discernible indication of the perfusion index.

2. The system of claim 1, wherein the first and second sensors include at least one of an accelerometer/force sensor, photoplethsmography sensor, an ECG sensor, one or more leads, a one to three axis accelerometer/force sensor, and an S1/S2 heart sound sensor.

3. The system of claim 1, further including a cardiac arrest detector.

4. The system of claim 1, further including a detector that detects the existence of PEA (Pulseless electrical activity).

5. The system of claim 1, wherein the processor is programmable to determine the presence of atrial fibrillation for those patients who have low EF (Ejection Fraction).

6. The system of claim 1 further including
a detector that detects a life threatening condition of a patient.

7. The system of claim 6, wherein the processor is programmable with parameter limits to allow for trigger level differences for alarms.

8. The system of claim 6, wherein the system is further arranged to determine Asystole.

9. A method of detecting a perfusion index of a cardiac pulse comprising:
sensing a first physiological or environmental parameter of a human patient core;
sensing a second physiological or environmental parameter of the human patient core;
determining, responsive to the first and second sensed parameters, a perfusion index ranging from 0 to 10 that reflects inadequate, marginal, or adequate blood perfusion to the core of the human patient torso; and
providing a discernible indication of the perfusion index.
